(19) United States
(12) Patent Application Publication
(10) Pub. No.: US 2003/0055255 A1
(43) Pub. Date: Mar. 20, 2003

(54) Synthesis of substituted imidazoles

(76) Inventors: Karen Marcantonio, New York, NY (US); Lisa Frey, Somerset, NJ (US); Doug Frantz, Downingtown, PA (US); Jerry A. Murry, New York, NY (US); Richard Tillyer, Cranford, NJ (US)

Correspondence Address: MERCK AND CO INC, P O BOX 2000, RAHWAY, NJ, 070650907

(21) Appl. No.: 10/177,861

(22) Filed: Jun. 20, 2002

Related U.S. Application Data
(60) Provisional application No. 60/299,490, filed on Jun. 20, 2001.

(51) Int. Cl. C 07D 4 1/14

(52) U.S. Cl. 546/193

(57) ABSTRACT

The present invention prepares imidazoles by the selective closure of a keto-amide to form the imidazolyl ring. In particular, the present invention selectively closes a keto-amide substituted with three rings to form a tri-substituted imidazole.

### BACKGROUND OF THE INVENTION

1. Field of the Invention

The present invention is directed to a method to prepare substituted imidazoles. In particular, this invention is directed to a method to prepare tri-cyclo substituted imidazoles.

2. Related Background

The present invention relates to prepare substituted tri-cyclo substituted imidazole compounds having cytokine inhibitory activity. Such substituted imidazole compounds are described in, for example, U.S. Pat. No. 5,717,100.

The substituted imidazole compounds demonstrate anti-cancer activity through the antagonism of the kinase, Raf. The raf genes code for a family of proteins, which can be oncogenically activated through N-terminal fusion, truncation or point mutations. RAF can be activated and undergoes rapid phosphorylation in response to PDGF, EGF, insulin, thrombin, endothelin, acidic FGF, CSF1 or TPA, as well as in response to oncoproteins v-fms, v-src, v-sis, Hras and polyoma middle T antigen. Antisense constructs which reduce cellular levels of c-Raf, and hence Raf activity, inhibit the growth of oncogene-transformed rodent fibroblasts in soft agar, while exhibiting little or no general cytotoxicity. Since inhibition of growth in soft agar is highly predictive of tumor responsiveness in whole animals, these studies suggest that the antagonism of RAF is an effective means by which to treat cancers in which RAF plays a role.

Examples of such cancers, where RAF is implicated through overexpression include cancers of the brain, genitourinary tract, lymphatic system, stomach, larynx and lung. More particularly, such examples include histiocytic lymphoma, lung adenocarcinoma and small cell lung cancers. Additional examples include cancers in which overexpression or activation of Raf-activating oncogenes (e.g., K-ras, erb-B) is observed. More particularly, such cancers include pancreatic and breast carcinoma.

Such substituted imidazoles also inhibit cytokines and the pathology, which is associated with diseases wherein cytokines are present in high levels. Cytokine mediated diseases refers to diseases or conditions in which excessive or unregulated production or activity of one or more cytokines occurs. Interleukin-1 (IL-1) and Tumor Necrosis Factor (TNF) are cytokines produced by a variety of cells that are involved in immunoregulation and other physiological conditions.

IL-1 is implicated in many disease states. Included among these diseases are rheumatoid arthritis, osteoarthritis, endotoxemia, toxic shock syndrome, acute and chronic inflammatory diseases, such as the inflammatory reaction induced by endotoxin or inflammatory bowel disease; tuberculosis, atherosclerosis, muscle degeneration, cachexia, psoriatic arthritis, Reiter's syndrome, rheumatoid arthritis, gout, traumatic arthritis, rubella arthritis and acute synovitis. Recent evidence also links IL-1 activity to diabetes.

Interleukin-1 has also been demonstrated to mediate a variety of biological activities thought to be important in immunoregulation and other physiological conditions. The known biological activities of IL-1 include the activation of T helper cells, induction of fever, stimulation of prostaglandin or collagenase production, neutrophil chemotaxis, induction of acute phase proteins and the suppression of plasma iron levels.

Excessive or unregulated tumor necrosis factor (TNF) production or activity has likewise been implicated in mediating or exacerbating rheumatoid arthritis, rheumatoid spondylitis, osteoarthritis, gouty arthritis, and other arthritic conditions, sepsis, septic shock, endotoxic shock, gram negative sepsis, toxic shock syndrome, adult respiratory distress syndrome, cerebral malaria, chronic pulmonary inflammatory disease, silicosis, pulmonary sarcosis, bone resorption diseases, reperfusion injury, graft v. host rejection, allograft rejections, fever and myalgia due to infection, cachexia secondary to infection or malignancy, cachexia secondary to acquired immune deficiency syndrome (AIDS), AIDS related complex (ARC), keloid formation, scar tissue formation, Crohn's disease, ulcerative colitis and pyresis.

Monokines, such as TNF, have also been shown to activate IRV replication in monocytes and/or macrophages. Therefore, inhibition of monokine production or activity aids in limiting HIV progression. TNF has been implicated in various roles with other viral infections, such as the cytomegalovirus (CMV), influenza virus, and the herpes virus.

Interleukin-6 (IL-6) is a cytokine effecting the immune system and hematopoiesis. It is produced by several mammalian cell types in response to agents such as IL-1, and is correlated with disease states such as angiofollicular lymphoid hyperplasia.

Interleukin-8 (IL-8) is a chemotactic factor first identified and characterized in 1987. Many different names have been applied to IL-8, such as neutrophil attractant/activation protein-1 (NAP-1), monocyte derived neutrophil chemotactic factor (MDNCF), neutrophil activating factor (NAF), and T-cell lymphocyte chemotactic factor. Like IL-1, IL-8 is produced by several cell types, including mononuclear cells, fibroblasts, endothelial cells and ketainocytes. Its production is induced by IL-1, TNF and by lipopolysaccharide (LPS). IL-8 stimulates a number of cellular functions in vitro. It is a chemoattractant for neutrophils, T-lymphocytes and basophils. It induces histamine release from basophils. It causes lysozomal enzyme release and respiratory burst from neutrophils, and it has been shown to increase the surface expression of Mac-1 (CD11b/CD18) on neutrophils without de novo protein synthesis.

There remains a need for more efficient synthesis of compounds which are effective for treating cancer in which RAF is implicated, as well as compounds which inhibit, suppress or antagonize the production or activity of cytokines such as IL-1, IL-6, IL-8 and TNF. Thus, there is a need for efficient synthesis of such cytokine inhibiting substituted imidazole compounds.

O. N. Popilin at al., Khim. Geterotsikl. Soedin., 9:1264-1265(1972) describes the synthesis of 1-substituted 2,5-diphenylimidazoles. C. F. Claiborne et al., Tetrahedron Lett., 39:8939-8942(1998) describes the synthesis of tetrasubstituted imidazoles. h. Alper et al., J. Org. Chem., 47:3593-3595(1982) describes the synthesis of amides by the reaction of Schiff bases with trialky or triarylboranes and carbon monoxide. V. Ambrogi et al., Eur. J. Med. Chem., 30:429-437(1995) describes the synthesis of 1- and 2-substituted imidazo[2,1-d][1,5]benzothiazepines. T. D. Gordon et al., Tetrahedron Lett., 34:1901-1904(1993) describes the synthesis of azole peptide mimetics.

K. V. P. Rao et al., Indian J. Chem., 24B:1120-1123(1985) describes the synthesis of 4H-imidazo-[2,1-c][1,4]benzoxaines. P. Schneiders et al., Chem. Ber., 106:2415-2417(1973) describes the synthesis of 4,4′,5,5′-tetrasubstituted di-2-imidazole derivatives. C. Zhang et al., Tetrahedron Lett., 37:751-754(1996) describes the synthesis of tetrasubstituted imidazoles. Y. G. Bal'on et al., J. Org. Chem. USSR, 86:1712-1715(1991) describes the synthesis of [alkyl(aryl)carbonylamino]arylmethyl aryl ketones for use in the synthesis of substituted azoles. K. Homma et al., Chem. Pharm. Bull., 45:1945-1954(1997) describes the synthesis of 2-aryl-4,5-dihydro-1H-thienyl[3,2-e]benzimidazoles.

H.-J. Kallmayer et al., Pharmazie, 46:328-331(1991) describes the synthesis of 2,2-dialkyl-naphthimidazoline quinones. h.-J. Kallmayer et al., Pharmazie, 46:247-249(1991) describes the synthesis of 2-dichloromethylnaphthimidazole quinones. h.-J. Kallmayer et al., Pharmazie, 46:502-505(1991) describes the synthesis of 1-aminopyrazolidines. E. M. Beccalli et al., Chemistry Lett., 659-662(1980) describes the synthesis of oxazolo- and imidazolo-phanes. T. W. von Geldem et al., J. Med. Chem., 39:957-967 describes the structure-activity profile of azole endothelin antagonists.

Nevertheless, there remains a need for an efficient method to synthesize substituted imidazole compounds.

### SUMMARY OF THE INVENTION

The present invention prepares imidazoles by the selective closure of a keto-amide to form the imidazolyl ring. In particular, the present invention selectively closes a keto-amide substituted with three rings to form a tri-substituted imidazole.

### DETAILED DESCRIPTION OF THE INVENTION

An example of a cytokine inhibiting substituted imidazole is

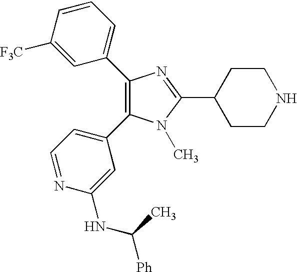

described in U.S. Pat. No. 5,717,100.

The present invention prepares tricyclo-substituted imidazoles by the selective closure of a keto-amide. The keto-amide is prepared by the reaction of a tosyl amide and an aldehyde or the aldehyde's cyanohydrin or acetal equivalent. A particular example is reacting the tosyl amide with 2-substituted-4-pyridine carboxaldehyde to form the keto-amide.

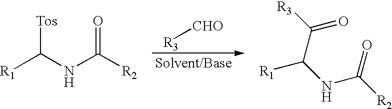

One Example is shown below:

### EXAMPLE

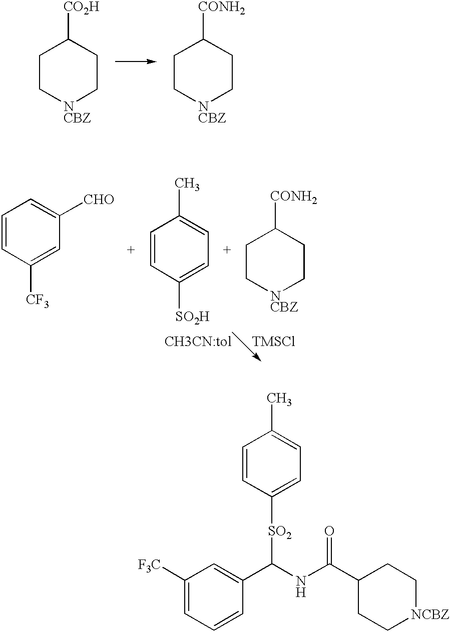

Preparation of Tosyl Amide:

To p-toluenesulfinic acid, sodium salt (2.7 kg, 15.2 mol, 1.5eq) and (Z)-isonipecotamide (4.0 kg, 15.2 mol, 1.5eq) was added acetonitrile (40L) and the contents stirred and placed under a positive pressure of nitrogen. To the resulting slurry was added 3-(trifluoromethyl)benzaldehyde (1.35L, 10.1 mol, 1.0 eq) in one portion. The mixture was then cooled to 10° C. using an ice bath. To the mixture was added chlorotrimethylsilane (TMSCl) (3.85L, 30.3 mol, 2.0 eq) slowly while maintaining an internal temperature below 25° C. After complete addition of the TMSCl, the reaction was allowed to warm to room temperature. The reaction was then monitored by HPLC until completion. To the heterogeneous mixture was added water (40L) and the resulting suspension was stirred for 120 min. The solids were isolated by filtration and the filter cake was washed with water (2×10L). The product was dried in a vacuum oven at 50° C. at 30 torr for 24 hours to give the product as a fine white solid.

¹H NMR (400 MHz, CDCl₃) δ 7.79-7.60 (m, 5H), 7.60-7.50 (t, J=7.5 Hz, 1H), 7.40-7.28 (m, 7H), 6.90 (d, J=11.3 Hz, 1H), 6.37 (d, J=11.3 Hz, 1H), 5.12 (s, 2H), 4.21-4.03 (br, 2H), 2.90-2.71 (br, 2H), 2.46 (s, 3H), 2.32 (m, 1H), 1.77-1.35 (m, 4H).

mp 158.1-158.7° C.

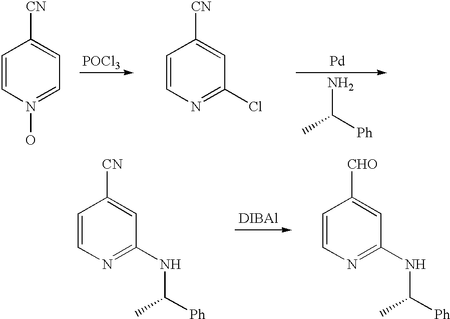

Preparation of 2-chloroisonicotinonitrile.

POCl₃ (9.0L, 97.4 mol) was added to 4-cyanopyridine N-oxide (3.0 kg, 24.98 mol) and the slurry slowly heated to 80° C. After 5h at 80° C., the reaction mixture became a clear solution and was warmed to 100° C. and aged for 24 h. After cooling to RT, the slurry was slowly added to pH 7 buffer (30L). During the addition, the temperature was kept at 15-35° C. and the pH was maintained at approx. 5-6 by addition of NaOH (9.6 N, 21L). The product was extracted into MTBE (18L) and the organic layer was treated with charcoal (Darco G-60, 600 g) for 4 h. The slurry was filtered over celite and washed with MTBE. The solvent was switched from MTBE to heptane and the slurry filtered to recover the product.

Analytical data consistent with that reported in the literature: Rokash, J. and Girard, Y. J. Heterocyclic Chem. 1978, 15, 683.

Preparation of 2-{[(1S)-1-phenylethyl]amino}-isonicotinonitrile.

(S)-α-methylbenzylamine (9.3L, 72.15 mol) was charged to a mixture of NaO^{t}Bu (1.53 kg, 15.87 mol), BINAP (180 g, 0.29 mol), and Pd(OAc)₂ (64.8 g, 0.29 mol) in toluene (30L). The mixture was heated to 50° C. and a solution of 2-chloro-4-cyano pyridine (2.0 kg, 14.43 mol) in toluene (10L) was added over 3 h. The mixture was stirred at 50° C. for 30 min and then cooled to room temperature. The reaction mixture was transferred into a 0-5° C. solution of THF (10L) and HOAc (25%, 25L), and the resulting two phase mixture was stirred vigorously for 30 min. The organic layer was washed with a mixture of THF (2L) and HOAc (25%, 4L). 2 N HCl (23L) and THF (7L) were added to the organic layer and stirred vigorously for 30 min. The organic layer was washed twice with 2 N HCl (2×12L). Toluene (14L) and THF (1L) were added to the acidic aqueous layer and the biphasic mixture was cooled to 5-10° C. and neutralized to pH 7 using NaOH (50%). The organic layer was stirred with charcoal (Darco G-60, 0.5 kg) and then filtered over celite and washed with toluene (7L). Solvent switching to 8% toluene in heptane and filtering the slurry afforded 2.64 kg (82%) of the title compound: ¹H NMR (DMSO-d₆) δ 8.09 (dd, J=0.6, 5.18, 1H), 7.58 (d, J=7.71, 1H), 7.36 (m, 2H), 7.29 (m, 2H), 7.18 (m, 1H), 6.82 (s, 1H), 6.73 (dd, J=1.4, 5.18, 1H), 5.03 (quintet, J=7.0, 2H), 1.43 (d, J=7.0, 3H) ppm; ¹³C NMR (DMSO-d₆) δ 158.4, 149.8, 145.5, 128.7, 127.0, 126.4 (2C), 119.9, 117.9, 112.1, 50.2, 23.7 ppm.

Preparation of 2-{[(1S)-1-phenylethyl]amino}isonicotinaldehyde.

Over 2 h, DIBAL (1.5 M tol, 16.8L, 25.2 mol) was added to a −20° C. solution of 2-{[(1S)-1-phenylethyl]aamino}-isonicotinonitrile (2.5 kg, 11.20 mol) in toluene (15.5L), maintaining the temperature below −10° C. After aging for 30 min, the reaction mixture was quenched into a 5-10° C. solution of 2N HCl (60.8L) and stirred vigorously for 2 h. The layers were separated and the pH of the aqueous layer was adjusted to pH=3 using 50% NaOH. A solution of sodium bisulfite (4.65 kg) in water (15L) was charged to the aqueous solution, affording rapid precipitation of the bisulfite adduct. After 4 h, the bisulfite adduct was filtered and washed with water (12.5L). The solid was dried in a 40° C. vacuum oven with a nitrogen sweep to yield the bisulfite adduct.

¹H NMR (DMSO-d₆) δ 9.11 (broad s, 1H), 7.73 (d, J=6.7, 1H), 7.42 (d, J=7.9, 2H), 7.36 (t, J=7.5, 2H), 7.28 (t, J=7.9, 1H), 7.21 (s, 1H), 6.96 (dd, J=1.3, 6.7, 1H), 5.10 (m, 1H), 5.04 (s, 1H), 1.52 (d, J=6.7, 3H);

¹³C NMR (DMSO-d₆) δ 156.6, 151.7, 142.4, 134.3, 129.1 (2C), 128.0, 126.5 (2C), 113.1, 111.3, 84.0, 51.2, 23.3.

X-Ray Powder confirmed that the product is crystalline.

EtOAc (29L) and the solid bisulfite adduct (4.12 kg) were added to a solution of KHCO₃ (9.3 kg) in water (30L). The tri-phasic mixture was stirred vigorously for 12 h and the aqueous layer was washed with EtOAc (12L) followed by toluene (12L). The organic layers were combined and solvent switched into toluene. THF was added to improve the solubility of the aldehyde. The final solution contained the aldehyde in 4:1 toluene:THF.

Aldehyde: ¹H NMR (CDCl₃) δ 9.83 (s, 1H), 8.27 (d, J=5.1, 1H), 7.36 (m, 4H), 7.25 (m, 1H), 6.94 (dd, J=1.2, 5.1, 1H), 6.26 (s, 1H), 5.43 (broad d, J=5.4, 1H), 4.85 (m, 1H), 1.59 (d, J=6.8, 3H);

¹³C NMR (CDCl₃) 8192.1, 159.0, 149.6, 144.0, 143.5, 128.9 (2C), 127.4, 125.9 (2C), 111.1, 106.5, 52.0, 24.1.

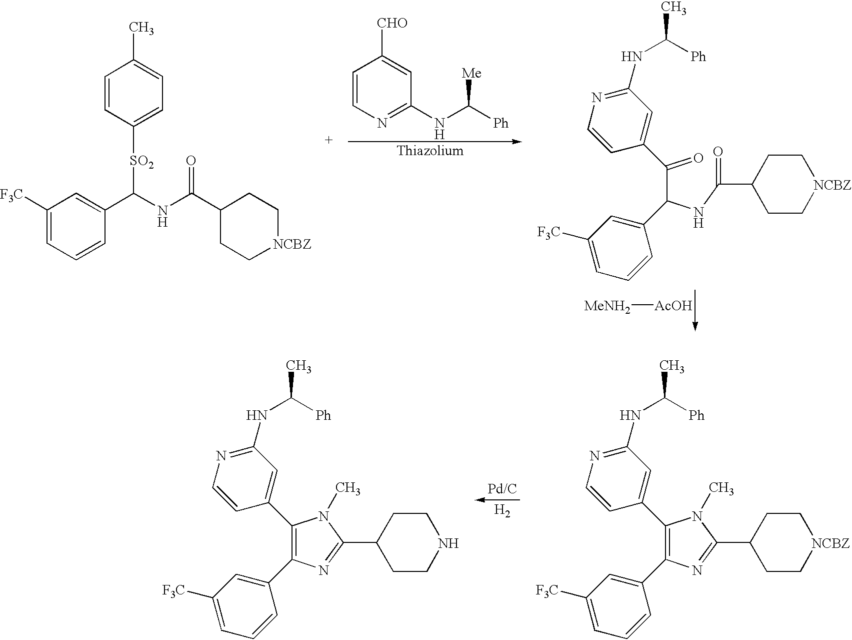

Procedure for the Preparation of the Keto-Amide:

To the tosyl-amide (3.7 kg, 6.4 mol, 1.2 eq) and the thiazolium catalyst (0.2 eq), purged with nitrogen, was added THF (35L) followed by the aldehyde (1.2 kg, 5.3 mol, 1.0 eq) and the resulting mixture stirred and heated to 50° C. Triethylamine (11.2L, 80 mol, 15 eq) was added in one portion and the corresponding reaction was monitored by HPLC analysis for consumption of the tosyl-amide. After the reaction was complete, it was cooled to 25° C. and water was added (10L) followed by toluene (10L). The resulting layers were separated and the organic layer was extracted with water (10L). The organic layer was washed with brine (10 mL), concentrated in vacuo to ½ volume at which time EtOH (40L) was added and concentration was continued. After complete concentration, 40L of fresh EtOH was added to the resulting thick oil to give an ethanolic solution of the keto-amide. Typically, this solution is carried on directly to the next step for imidazole formation. However, if desired, the corresponding keto-amide can be isolated by evaporation to dryness to give a light yellow amorphous solid as a 1:1 mixture of diastereomers.

¹H NMR (CDCl₃) δ 8.16 (d, J=4.5 Hz, 1H), 7.57-7.16 (m, 14H), 6.98-6.85 (m, 2H), 6.62 and 6.58 (2s, 1H), 6.28 and 6.25 (2d, J=4.5 Hz, 1H), 5.37 and 5.26 (2d, J=6.8 Hz, 1H), 5.13 (s, 2H), 4.79-4.64 (m, 1H), 4.30-4.10 (br, 2H), 2.90-2.76 (br, 2H), 2.38-2.28 (m, 1H), 1.90-1.56 (m, 4H), 1.56 and 1.52 (2d, J=6.8 Hz, 3H);

mp 74.1-75.8° C.

Procedure for the Preparation of the Tetra-Substituted Imidazole:

The ethanolic solution of the keto-amide obtained in the previous procedure (40L) was transferred to a vessel containing methyl ammonium acetate (106 mol, 20 eq) and the resulting solution was heated to reflux for 3 h at which time HPLC showed that the reaction was complete. The reaction mixture was concentrated to ½ volume and cooled to ambient temperature at which point the product crystallized out of solution. 50% aqueous ethanol was added to the reaction mixture and the product filtered. The solid was washed with 50% aq EtOH and dried in a drying oven. This solid matched the previously reported analytical data in U.S. Pat. No. 5,717,100 for this compound in all respects.

What is claimed is:

1. A method of forming a reaction product mixture substantially containing 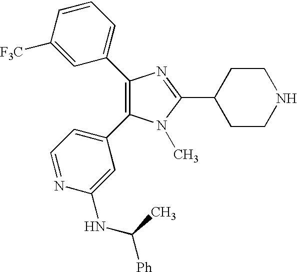
said method comprising:
reacting 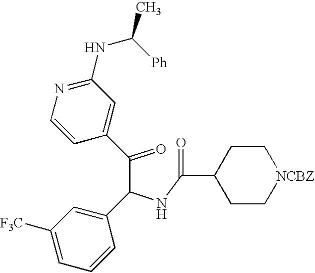
in the presence of an effective amount of methyl ammonium acetate.
